(19) United States
(12) Patent Application Publication
(10) Pub. No.: US 2009/0264616 A1
(43) Pub. Date: Oct. 22, 2009

(54) Cyclodipeptide Synthetases and Their Use for Synthesis of Cyclo(Leu-Leu) Cyclodipeptide

(75) Inventors: Muriel Gondry, Limours (FR); Robert Thai, Nozay (FR); Pascal Belin, Igny (FR); Roger Genet, Limours (FR); Jean-Luc Pernodet, Cachan (FR)

Correspondence Address: THE NATH LAW GROUP, 112 South West Street, Alexandria, VA, 22314, US

(73) Assignees: COMMISSARIAT A L'ENERGIE ATOMIQUE, Paris (FR); KYOWA HAKKO BIO CO., LTD., Tokyo (JP); UNIVERSITE PARIS SUD 11, Orsay Cedex (FR); CENTRE NATIONAL DE LA RECHERCHE SEIENTIFIQUE, Paris (FR)

(21) Appl. No.: 12/226,661

(22) PCT Filed: Apr. 26, 2006
(86) PCT No.: PCT/IB2006/001849

§ 371 Date: Feb. 24, 2009

(51) Int. Cl. C07K 5/06; C12N 9/00; C07H 21/04; C12N 15/63; C12P 21/06

(52) U.S. Cl. 530/300; 435/183; 536/23.2; 435/320.1; 435/68.1

(57) ABSTRACT

Isolated, natural or synthetic polynucleotides and the polypeptides encoded by said polynucleotides, that are involved in the synthesis of cyclodipeptides, the recombinant vectors comprising said polynucleotides or any substantially homologous polynucleotides, the host cell modified with said polynucleotides or said recombinant vectors and also methods for in vitro and in vivo synthesizing cyclo(Leu-Leu) cyclodipeptide and its derivatives and applications thereof.

The present invention relates to isolated, natural or synthetic polynucleotides and to the polypeptides encoded by said polynucleotides, that are involved in the synthesis of cyclodipeptides, to the recombinant vectors comprising said polynucleotides or any substantially homologous polynucleotides, to the host cell modified with said polynucleotides or said recombinant vectors and also to methods for in vitro and in vivo synthesizing cyclo(Leu-Leu) cyclodipeptide and its derivatives.

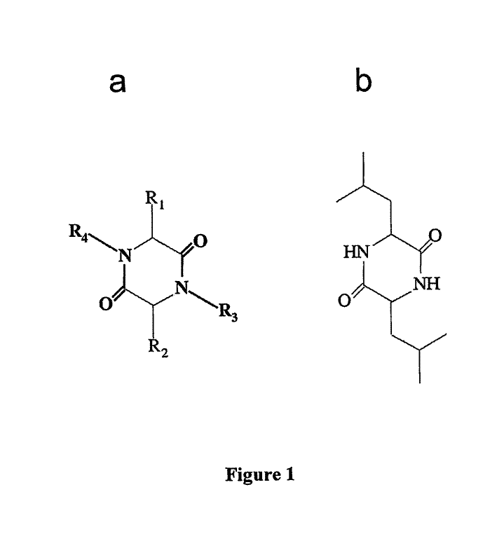

For the purposes of the present invention, the term “diketopiperazine derivatives” or “DKP” or “2,5-DKP” or “cyclic dipeptides” or “cyclodipeptides” or “cyclic diamino acids” is intended to mean molecules having a diketopiperazine (piperazine-2,5-dione or 2,5-dioxopiperazine) ring. In the particular case of α,β-dehydrogenated cyclodipeptide derivatives, the substituent groups R1 and R2 are α,β-unsaturated amino acyl side chains (FIG. 1). Such derivatives are hereafter referred to as “Δ” derivatives.

The DKP derivatives constitute a growing family of compounds that are naturally produced by many organisms such as bacteria, yeast, filamentous fungi and lichens. Others have also been isolated from marine organisms, such as sponges and starfish. An example of these derivatives: cyclo(L-His-L-Pro), has been shown to be present in mammals.

The DKP derivatives display a very wide diversity of structures ranging from simple cyclodipeptides to much more complex structures. The simple cyclodipeptides constitute only a small fraction of the DKP derivatives, the majority of which have more complex structures in which the main ring and/or the side chains comprise many modifications: introduction of carbon-based, hydroxyl, nitro, epoxy, acetyl or methoxy groups, and also the formation of disulfide bridges or of hetero-cycles. The formation of a double bond between two carbons is also quite widespread. Certain derivatives, of marine origin, incorporate halogen atoms.

Useful biological properties have already been demonstrated for some of the DKP derivatives. Bicyclomycin (Bicozamine™) is an antibacterial agent used as food additive to prevent diarrhea in calve and swine (Magyar et al., J. Biol. Chem, 1999, 274, 7316-7324). Gliotoxin has immunosuppressive properties which were evaluated for the selective ex vivo removal of immune cells responsible for tissue rejection (Waring et al., Gen. Pharmacol., 1996, 27, 1311-1316). Several compounds such as ambewelamides, verticillin and phenylahistin exhibit antitumour activities involving various mechanisms (Chu et al., J. Antibiot. (Tokyo), 1995, 48, 1440-1445; Kanoh et al., J. Antibiot. (Tokyo), 1999, 52, 134-141; Williams et al., Tetrahedron Lett., 1998, 39, 9579-9582).

Many others like albonoursin produced by Streptomyces noursei, display antimicrobial activities (Fukushima et al., J. Antibiot. (Tokyo), 1973, 26, 175-176). Cyclo(Tyr-Tyr) and cyclo(Tyr-Phe) were shown to be potential cardioactive agents: cyclo(Tyr-Tyr) being a potential cardiac stimulant and cyclo(Tyr-Phe) being a cardiac inhibitor (Kilian et al., Pharmazie, 2005, 60, 305-309). These two cyclo-dipeptides were also tested as receptor interacting agents and the two compounds were found to exhibit significant binding to opioid receptors (Kilian et al., 2005, precited). Moreover, they were evaluated as antineoplastic agents and cyclo(Tyr-Phe) was shown to induce growth inhibition of three different cultured cell lines (Kilian et al., 2005, precited). It has been described that the cyclo(ΔAla-L-Val) produced by Pseudomonas aeruginosa could be involved in interbacterial communication signals (Holden et al., Mol. Microbiol., 1999, 33, 1254-1266). Other compounds are described as being involved in the virulence of pathogenic microorganisms or else as binding to iron or as having neurobiological properties (King et al., J. Agr. Food Chem., 1992, 40, 834-837; Sammes, Fortschritte der Chemie Organischer Naturstoffe, 1975, 32, 51-118; Alvarez et al., J. Antibiot., 1994, 47, 1195-1201).

Although the number of known DKPs is increasing steadily, biosynthesis pathways of these compounds are still largely unexplored, leading to little knowledge regarding their synthesis.

In several cases reported so far, the formation of DKPs occurs spontaneously from linear dipeptides for which the cis-conformation of the peptide bond is favoured by the presence of an N-alkylated amino acid or a proline residue. Such spontaneous cyclisation has also been observed in the course of non ribosomal peptide synthesis of gramicidin S and tyrocidine A in Bacillus brevis, due to the instability of the thioester linkage during peptide elongation on peptide synthetase megacomplexes (Schwarzer et al., Chem. Biol, 2001, 8, 997-1010). Thus, in all of the known mechanisms of spontaneous DKP formation, the primary structure of the precursor dipeptide, in particular the conformation of its peptide bond, appears to be a fundamental requirement for the formation of the DKP ring to take place and for the process to result in the production of the final DKP derivative.

However, such a spontaneous cyclisation reaction cannot account for the biosynthesis of the large majority of DKP derivatives that do not contain a proline residue or an N-alkylated residue.

Known methods for producing DKP-derivatives include chemical synthesis, extraction from natural producer organisms and also enzymatic methods:
Chemical methods can be used for synthesizing DKP derivatives (Fischer, J. Pept. Sci., 2003, 9, 9-35) but they are considered to be disadvantageous in respect of cost and efficiency as they often necessitate the use of protected amino acyl precursors and lead to the loss of stereochemical integrity. Moreover, they are not environment-friendly methods as they use large amounts of organic solvents and the like.
Extraction from natural producer organisms can be used but the productivity remains low because the contents of desired DKP-derivatives in natural products are often low.
Enzymatic methods, i.e. methods utilizing enzymes either in vivo (e.g. culture of microorganisms expressing cyclodipeptide-synthesizing enzymes or microorganism cells isolated from the culture medium) or in vitro (e.g. purified cyclodipeptide-synthesizing enzymes) can be used. Enzymes known to produce cyclodipeptides are non-ribosomal peptide synthetases (hereinafter referred to as NRPS) (Gruenewald et al., Appl. Environ. Microbiol., 2004, 70, 3282-3291) and AlbC which is a cyclodipeptide synthetase (CDS) (Lautru et al., Chem. Biol., 2002, 9, 1355-1364; International Application WO 2004/000879):
the enzymatic method utilizing NRPS has already been reported to produce a specific cyclodipeptide. The two genes coding for the bimodular complex TycA/TycB1 from Bacillus brevis (Mootz and Marahiel, J. Bacteriol., 1997, 179, 6843-6850) were coexpressed in Escherichia coli and gave rise to the production of cyclo(DPhe-Pro) (Gruenewald et al., Appl. Environ. Microbiol., 2004, 70, 3282-3291). The cyclodipeptide was stable, not toxic to E. coli and secreted in the culture medium. However, the methods utilizing NRPS appear essentially restricted to the production of cyclodipeptides containing N-alkylated residues. Indeed, the formation of DKP derivatives occurs spontaneously from linear dipeptides for which the cis conformation of the peptide bond is favored by the presence of prolyl residues (Walzel et al., Chem. Biol., 1997, 4, 223-230; Schwarzer et al., Chem. Biol., 2001, 8, 997-1010) or N-methylated residues (Healy et al, Mol. Microbiol., 2000, 38, 794-804). Moreover, the methods utilizing NRPS are difficult to implement: NRPS being large multimodular enzyme complexes, they are not easy to manipulate both at the genetic or biochemical levels.
the enzymatic method utilizing AlbC was also described to produce specific cyclodipeptides. The expression of AlbC from Streptomyces noursei by heterologous hosts Streptomyces lividans TK21 or E. coli led to the production of two cyclodipeptides, cyclo(Phe-Leu) and cyclo(Phe-Phe) that were secreted in the culture medium (Lautru et al., Chem. Biol., 2002, 9, 1355-1364). AlbC catalyzes the condensation of two amino acyl derivatives to form cyclodipeptides, containing or not containing N-alkylated residues, by an unknown mechanism. This unambiguously shows that a specific enzyme unrelated to non ribosomal peptide synthetases can catalyze the formation of DKP derivatives: AlbC is the first example of an enzyme that is directly involved in the formation of the DKP motif.

Furthermore the obtained cyclo(Phe-Leu) cyclodipeptide may be transformed into a cyclo(α,β-dehydro-dipeptide), i.e. albonoursin, or cyclo(ΔPhe-ΔLeu), an antibiotic produced by Streptomyces noursei, in the presence of cyclic dipeptide oxydase (CDO) which specifically catalyzed the formation of albonoursin, in a two-step sequential reaction starting from the natural substrate cyclo(L-Phe-L-Leu) leading first to cyclo(ΔPhe-L-Leu) and finally to cyclo(ΔPhe-ΔLeu) corresponding to albonoursin (Gondry et al., Eur. J. Biochem., 2001, 268, 1712-1721). Said CDO may also transform various cyclodipeptides into α,β-dehydrodipeptides (Gondry et al., Eur. J. Biochem., 2001, precited).

The DKP derivatives exhibit various biological functions, making them useful entities for the discovery and development of new drugs, food additives and the like. Accordingly, it is necessary to be able to have large amounts of these compounds available.

An understanding of the pathways for the natural synthesis of the diketopiperazine derivatives could enable a reasoned genetic improvement in the producer organisms, and would open up perspectives for substituting or improving the existing processes for synthesis (via chemical or biotechnological pathways) through the optimization of production and purification yields. In addition, modification of the nature and/or of the specificity of the enzymes involved in the biosynthetic pathway for the diketopiperazine derivatives could result in the creation of novel derivatives with original molecular structures and with optimized biological properties.

The Inventors have now identified new cyclodipeptide synthesizing enzymes (or cyclodipeptide synthetases or CDS), said enzymes sharing at least 34% of identity or at least 56% of similarity with at least one of the polypeptide of the sequences SEQ ID No: 2, SEQ ID No: 4, SEQ ID No: 6 and SEQ ID No: 8, and said enzymes being able to catalyse the specific formation of cyclo(Leu-Leu) cyclo-dipeptide.

These percentages of sequence identity and sequence similarity defined herein were obtained using the BLAST program (blast2seq, default parameters) (Tatutsova and Madden, FEMS Microbiol Lett., 1999, 174, 247-250).

Said percentages derived from the comparison of the full-length sequences SEQ ID No: 2, SEQ ID No: 4, SEQ ID No: 6 and SEQ ID No: 8 one with another, as described in Table V; preferably said percentages derived by calculating them on an overlap representing a percentage of length of said sequences as specified in Table V.

The polypeptide of SEQ ID No: 2 corresponds to the polypeptide YvmC_{sub} of Bacillus subtilis subsp. subtilis strain 168 (GenBank accession number Acc. n^{o} CAB 15512). The polypeptide of SEQ ID No 4 corresponds to the polypeptide Plu0297 of Photorhabdus luminescens subsp. laumondii TTO1 (Genbank Acc. n^{o} Q7N9M5). The polypeptide of sequence SEQ ID No 6 corresponds to the polypeptide YvmCₜₕᵤ of Bacillus thuringiensis serovar israelensis strain 1884, and shares 94% of identity and 98% of similarity with the polypeptide RBTH_{—}07362 of Bacillus thuringiensis serovar israelensis ATCC 35646 (Genbank Acc. n^{o} EA057133). The polypeptide of SEQ ID No 8 corresponds to the polypeptide YvmC_{lic} of Bacillus licheniformis ATCC 14580 (Genbank Acc. n^{o} AAU25020). The informations available on the different databases concern hypothetical proteins which were not isolated and for which no function has been defined.

Thus one object of the invention is the use of an enzyme sharing at least 34% of identity or at least 56% of similarity, with at least one of the polypeptides of sequences SEQ ID No 2, SEQ ID No 4, SEQ ID No 6 and SEQ ID No 8, for the synthesis of a cyclo(Leu-Leu) cyclodipeptide.

Preferably, said cyclodipeptide synthetase shares at least 60% of identity or at least 70% of similarity with at least two of the polypeptides having the sequences SEQ ID No 2, SEQ ID No 4, SEQ ID No 6 and SEQ ID No 8, even more preferably with three of the polypeptides having the sequences SEQ ID No 2, SEQ ID No 4, SEQ ID No 6 and SEQ ID No 8.

Advantageously, said cyclodipeptide synthetase shares at least 60% of identity or 70% of similarity, with the three polypeptides having the sequences SEQ ID NO 2, SEQ ID No 6 and SEQ ID No 8.

Preferably, said cyclodipeptide synthetase shares at least 60% of identity and more preferably at least 65% of identity or at least 70% of similarity and preferably at least 75% of similarity, with at least one of the polypeptides having the sequences SEQ ID No 2, SEQ ID No 4, SEQ ID No 6 and SEQ ID No 8.

More preferably, said cyclodipeptide synthetase shares at least 70% of identity and even more preferably at least 75% of identity or at least 75% of similarity even more preferably at least 80% of similarity, with at least one of these four sequences.

Advantageously, said cyclodipeptide synthetase shares at least 80% of identity and more advantageously at least 85% of identity or at least 85% of similarity, with at least one of those four sequences.

It is preferred that said cyclodipeptide synthetase enzyme shares at least 90% of identity or at least 90% of similarity, even more preferably at least 95% of identity or at least 95% of similarity, advantageously at least 98% of identity or of similarity, and even more advantageously at least 99% of identity or of similarity, with at least one of the sequences SEQ ID No 2, SEQ ID No 4, SEQ ID No 6 and SEQ ID No 8.

More preferably said enzyme is selected from the group consisting of the polypeptides of sequences SEQ ID No 2, SEQ ID No 4, SEQ ID No 6 and SEQ ID No 8.

The object of the present invention is also an isolated cyclodipeptide synthetase, characterized in that:
it has the ability to produce cyclo(Leu-Leu) cyclodipeptide from two Leu amino acids and
it comprises a polypeptide sequence having at least 34% of identity or at least 56% of similarity, with at least one of the polypeptides of sequences SEQ ID No 2, SEQ ID No 4, SEQ ID No 6 and SEQ ID No 8 and preferably at least 60% of identity or at least 70% of similarity with at least two of the polypeptides having the sequences SEQ ID No 2, SEQ ID No 4, SEQ ID No 6 and SEQ ID No 8,

Said isolated cyclodipeptide synthetase is preferably selected in the group consisting of the sequences SEQ ID No 2, SEQ ID No 4, SEQ ID No 6 and SEQ ID No 8.

Another object of the invention is the use of a polynucleotide selected from:

a) a polynucleotide encoding a cyclodipeptide synthetase as defined above;

b) a complementary polynucleotide of the polynucleotide a);

c) a polynucleotide which hybridizes to polynucleotide a) or b) under stringent conditions, for the synthesis of a cyclo(Leu-Leu) cyclodipeptide.

Advantageously, said polynucleotide is selected from the group consisting of the polynucleotides of sequences SEQ ID No 1, SEQ ID No 3, SEQ ID No 5 and SEQ ID No 7. The polynucleotides of sequences SEQ ID No 1, SEQ ID No 3, SEQ ID No 5 and SEQ ID No 7 encode respectively the polypeptides of sequences SEQ ID No 2, SEQ ID No 4, SEQ ID No 6 and SEQ ID No 8.

The term “hybridize(s)” as used herein refers to a process in which polynucleotides hybridize to the recited nucleic acid sequence or parts thereof. Therefore, said nucleic acid sequence may be useful as probes in Northern or Southern Blot analysis of RNA or DNA preparations, respectively, or can be used as oligo-nucleotide primers in PCR analysis dependent on their respective size. Preferably, said hybridizing polynucleotides comprise at least 10, more preferably at least 15 nucleotides while a hybridizing polynucleotide of the present to be used as a probe preferably comprises at least 100, more preferably at least 200, or most preferably at least 500 nucleotides.

It is well known in the art how to perform hybridization experiments with nucleic acid molecules, i.e. the person skilled in the art knows what hybridization conditions she/he has to use in accordance with the present invention. Such hybridization conditions are referred to in standard text books such as Sambrook et al., Molecular Cloning: A Laboratory Manual; Cold Spring Harbor Laboratory Press, 2^{nd} edition 1989 and 3^{rd} edition 2001; Gerhardt et al.; Methods for General and Molecular Bacteriology; ASM Press, 1994; Lefkovits; Immunology Methods Manual: The Comprehensive Sourcebook of Techniques; Academic Press, 1997; Golemis; Protein-Protein Interactions: A Molecular Cloning Manual; Cold Spring Harbor Laboratory Press, 2002 and other standard laboratory manuals known by the person skilled in the Art or as recited above. Preferred in accordance with the present inventions are stringent hybridization conditions.

“Stringent hybridization conditions” refer, e.g. to an overnight incubation at 42° C. in a solution comprising 50% formamide, 5×SSC (750 mM NaCl, 75 mM sodium citrate), 50 mM sodium phosphate (pH 7.6), 5×Denhardt's solution, 10% dextran sulfate, and 20 μg/ml denatured, sheared salmon sperm DNA, followed e.g. by washing the filters in 0.2×SSC at about 65° C.

Also contemplated are nucleic acid molecules that hybridize at low stringency hybridization conditions. Changes in the stringency of hybridization and signal detection are primarily accomplished through the manipulation of formamide concentration; salt conditions, or temperature. For example, lower stringency conditions include an overnight incubation at 37° C. in a solution comprising 6×SSPE (20×SSPE=3 mol/l NaCl; 0.2 mol/l NaH₂PO₄; 0.02 mol/l EDTA, pH 7.4), 0.5% SDS, 30% formamide, 100 μg/ml salmon sperm blocking DNA; followed by washes at 50° C. with 1×SSPE, 0.1% SDS.

In addition, to achieve even lower stringency, washes performed following stringent hybridization can be done at higher salt concentrations (e.g. 5×SSC). It is of note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations.

Another object of the invention is an isolated polynucleotide selected from:

a) a polynucleotide encoding a cyclodipeptide synthetase as defined above;

b) a complementary polynucleotide of the polynucleotide a);

c) a polynucleotide which hybridizes to polynucleotide a) or b) under stringent conditions.

Advantageously, said isolated polynucleotide is selected from the group consisting of the polynucleotides of sequences SEQ ID No 1, SEQ ID No 3, SEQ ID No 5 and SEQ ID No 7.

Said isolated polynucleotides can be obtained from DNA libraries, particularly from microorganism DNA libraries. For example the polynucleotide having the sequence SEQ ID No 1 can be obtained from a Bacillus subtilis DNA library, the polynucleotide having the sequence SEQ ID No 3 can be obtained from a Photorhabdus luminescens DNA library, the polynucleotide having the sequence SEQ ID No 5 can be obtained from a Bacillus thuringiensis DNA library, the polynucleotide having the sequence SEQ ID No 7 can be obtained from a Bacillus licheniformis DNA library. Said polynucleotides can also be obtained by means of a polymerase chain reaction (PCR) carried out on the total DNA of the respective microorganisms, or can be obtained by RT-PCR carried out on the total RNA of the same microorganism.

Another object of the invention is a recombinant vector characterized in that it comprises a polynucleotide as defined above.

Said vector may be any known vector of the prior art, and is preferably an expression vector. As vectors that can be used according to the invention, mention may in particular be made of plasmids, cosmids, bacterial artificial chromosomes (BACs), integrative elements of actinobacteria, viruses or bacteriophages.

Said vector may also comprise any regulatory sequences required for the replication of the vector and/or the expression of the polypeptide encoded by the polynucleotide (promoter, termination sites, etc. . . . ).

Another object of the invention is a modified host cell into which a polynucleotide as defined above or a recombinant vector of the invention, has been introduced.

Such a modified host cell may be any known heterologous expression system using prokaryotes or eukaryotes as hosts, and is preferably a prokaryotic cell. By way of example, mention may be made of animal or insect cells, and preferably of a microorganism and in particular a bacterium such as Escherichia coli.

The introduction of the polynucleotide or the recombinant vector according to the invention into the host cell to be modified can be carried out by any known method, such as, for example, transfection, infection, fusion, electroporation, microinjection or else biolistics.

Another object of the present invention is the use of a recombinant vector or a modified host cell of the invention, for the synthesis of cyclo(Leu-Leu) cyclodipeptide.

In another aspect, the present invention relates to a method for the synthesis of cyclo(Leu-Leu) cyclodipeptide characterized in that it comprises the steps of:

(1) incubating leucine, under suitable conditions, with a cyclodipeptide synthetase enzyme as defined above, and

(2) recovering the cyclo(Leu-Leu) cyclodipeptide thus obtained.

The term “suitable conditions” is preferably intended to mean the appropriate conditions (concentrations, pH, buffer, temperature, time of reaction, etc. . . . ) under which the amino acids and the cyclodipeptide synthetase are incubated, to allow the synthesis of cyclodipeptides in an appropriate buffer.

An example of an appropriate concentration of amino acids and cyclodipeptide synthetase is the following: leucine at a concentration of between 0.1 mM and 100 mM, preferably of between 1 mM and 10 mM; the cyclodipeptide synthetase as defined above (e.g. polypeptide of a sequence selected from SEQ ID No 2, SEQ ID No 4, SEQ ID No 6 and SEQ ID No 8) is at a concentration of between 0.1 nM and 100 μM, preferably of between 1 μM and 100 μM.

An example of an appropriate buffer is 100 mM Tris-HCl, containing 150 mM NaCl, 10 mM ATP, 20 mM MgCl₂, supplemented with a soluble prokaryote cell extract.

Appropriate pH is ranging between 6 and 8, appropriate temperature is ranging between 20 and 40° C., and appropriate reaction time is ranging between 12 and 24 hours.

Therefore, according to a preferred embodiment of carrying out said method, step (1) is performed in presence of Leu at a concentration between 0.1 mM and 100 mM, preferably of 1 mM to 10 mM, a cyclodipeptide synthetase as defined hereabove at a concentration between 0.1 nM and 100 μM, preferably of 1 μM to 100 μM, in a buffer at a pH between 6 and 8, and containing a soluble extract of prokaryote cells such as E. coli or Streptomyces cells which do not produce cyclodipeptide synthetase.

α,β-dehydrogenated cyclo(Leu-Leu) cyclopeptide derivative may be obtained from the hereabove described cyclodipeptide, according to the method described in Gondry et al. (Eur. J. Biochem., 2001, precited) or in the International PCT Application WO 2004/000879.

For example, an amount of 5 10⁻³ units of CDO is added to the buffer used according to the method described above. One unit of CDO was defined as the amount catalyzing the formation of 1 mol of cyclo(ΔPhe-His) per min under standard assay conditions (Gondry et al., Eur. J. Biochem., 2001, 268, 4918-4927).

Therefore, according to a preferred embodiment of said method it comprises:

(1′) incubating leucine, under suitable conditions, with a cyclodipeptide synthetase enzyme as defined above and a purified CDO, and

(2′) recovering the α,β-dehydrogenated cyclopeptide.

According to another preferred embodiment of the method for the synthesis of cyclo(Leu-Leu) cyclodipeptide or α,β-dehydrogenated derivative thereof, a preliminary step (P) consisting in the use of a polynucleotide as defined above, for synthesizing cyclodipeptide synthetase, is performed before step (1).

The methods of synthesis of cyclo(Leu-Leu) cyclodipeptide or α,βdehydrogenated derivative thereof may be carried out in any suitable biological system, particularly in a host such as, for example, a microorganism, for instance a bacterium such as Escherichia coli or Streptomyces lividans, or any known heterologous expression system using prokaryotes or eukaryotes as hosts, or even in vitro acellular systems.

Another object of the invention is a method for producing cyclo(Leu-Leu) cyclodipeptide, comprising the following steps:

(1′) culturing a modified host cell of the invention, as defined above in appropriate culture conditions for said host cell, and

(2′) recovering the cyclo(Leu-Leu) cyclodipeptide from the culture medium.

α,β-dehydrogenated cyclo(Leu-Leu) cyclodipeptide derivative may be obtained from the cyclo(Leu-Leu) cyclodipeptide, according to the method described in Gondry et al. (Eur. J. Biochem., 2001, precited) or in the International PCT Application WO 2004/000879, in the following conditions:

(1′) culturing a modified host cell as defined here above in appropriate culture conditions for said host cell,

(1″) incubating the cyclo(Leu-Leu) cyclodipeptide obtained from step (1′) with a purified CDO, and

(2″) recovering the α,β-dehydrogenated cyclo(Leu-Leu) cyclodipeptide derivative from the culture medium.

The conditions for using CDO are the same than those described above.

The recovering of the cyclo(Leu-Leu) cyclodipeptide or of the α,β-dehydrogenated derivative thereof can be carried out directly from synthesis by means of liquid-phase extraction techniques or by means of precipitation, or thin-layer or liquid-phase chromatography techniques, in particular reverse-phase HPLC, or any method suitable for purifying peptides, one known to those skilled in the art.

Another object of the instant invention is also α,β-dehydrogenated cyclo(Leu-Leu) derivatives: cyclo(ΔLeu-Leu) or cyclo(ΔLeu-ΔLeu).

Besides the above provisions, the invention also comprises other provisions which will emerge from the following description, which refers to examples of implementation of the invention and also to the attached drawings, in which:

FIG. 1(a) represents the structure of piperazine-2,5-dione cycle. The cis-amide bond is in bold. FIG. 1(b) represents the structure of cyclo(Leu-Leu).

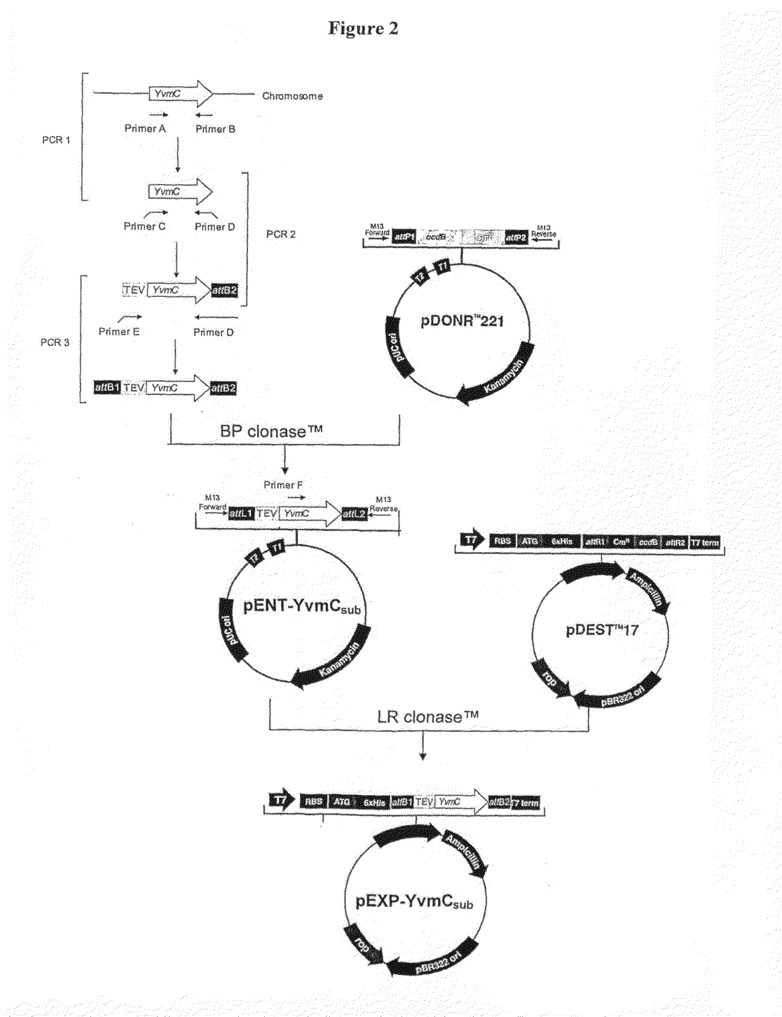

FIG. 2 illustrates the cloning strategy for the construction of the expression vector pEXP-YvmC_{sub}.

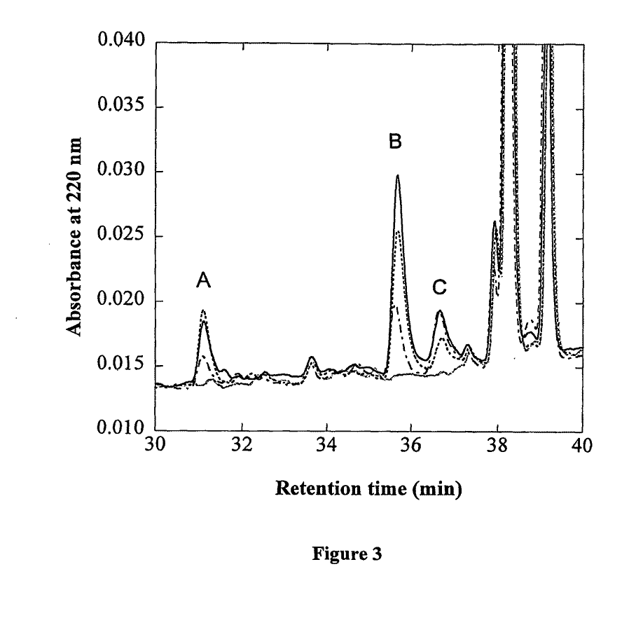

FIG. 3 represents the HPLC analysis of the culture media of E. coli BL21 AI cells expressing the YvmC_{sub}, YvmCₜₕᵤ or YvmC_{lic} proteins. Culture supernatants of cells transformed with: [symbol] pEXP-YvmC_{sub}, [symbol] pEXP-YvmCₜₕᵤ, [symbol] pEXP-YvmC_{lic}, and as a control, [symbol] empty pQE60,

were analyzed by RP-HPLC. The chromatograms were recorded at 220 nm.

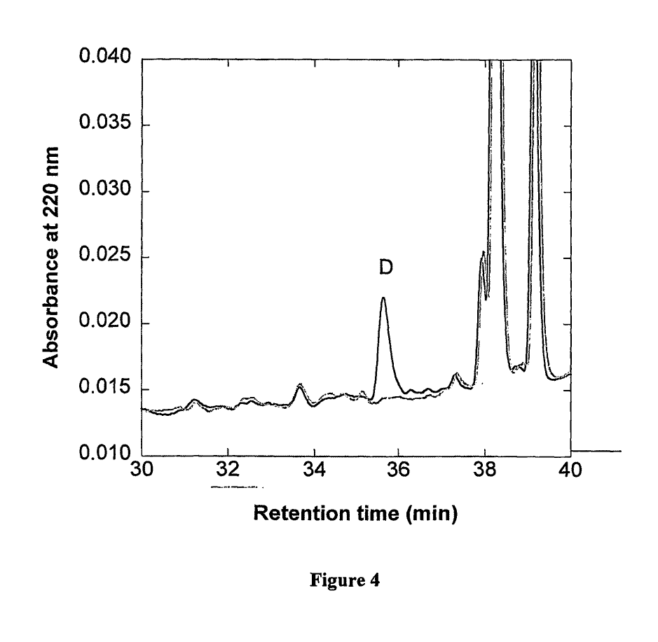

FIG. 4 illustrates the HPLC analysis of the culture media of E. coli BL21 AI cells expressing the Plu0297 protein. Culture supernatants of cells transformed with: [symbol] pEXP-Plu0297, and as a control [symbol] empty pQE60,

were analyzed by RP-HPLC. The chromatograms were recorded at 220 nm.

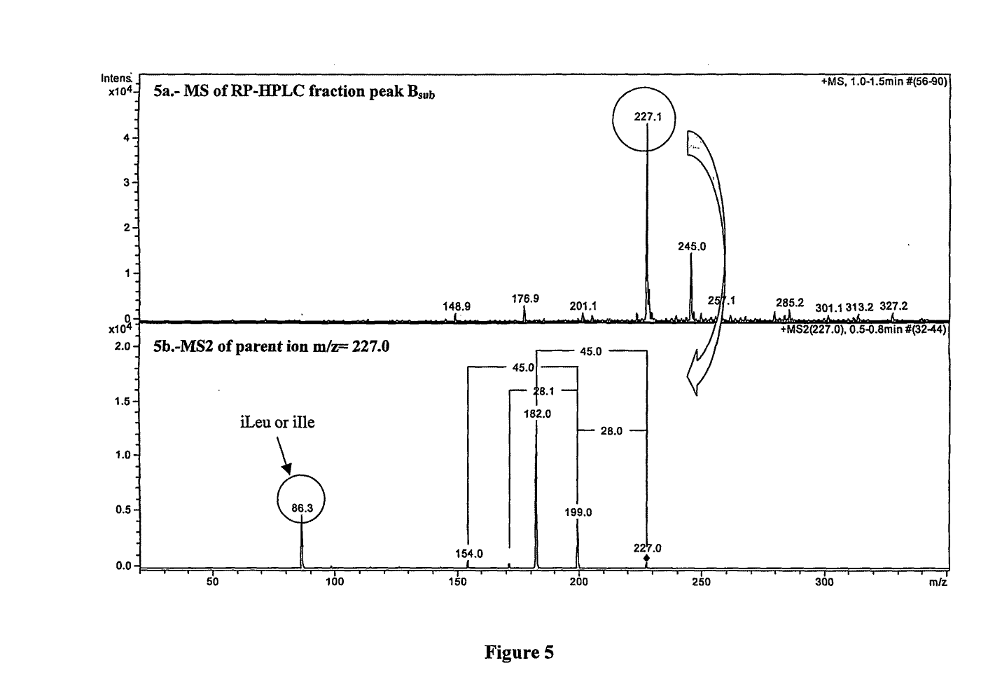

FIG. 5 illustrates the MS (a) and MSMS (b) spectra of collected fraction of peak B_{sub}. Collected fraction was directly infused into the mass spectrometer and full scan MS acquired on line (FIG. 5a). Main m/z peak at 227.0±0.1 was isolated as parent ion and subjected to MSMS fragmentation giving rise to a daughter ion spectrum (FIG. 5b). Encircled peak m/z at 86.3 matches to immonium ion of leucine or isoleucine, respectively referred to as iLeu and iIle.

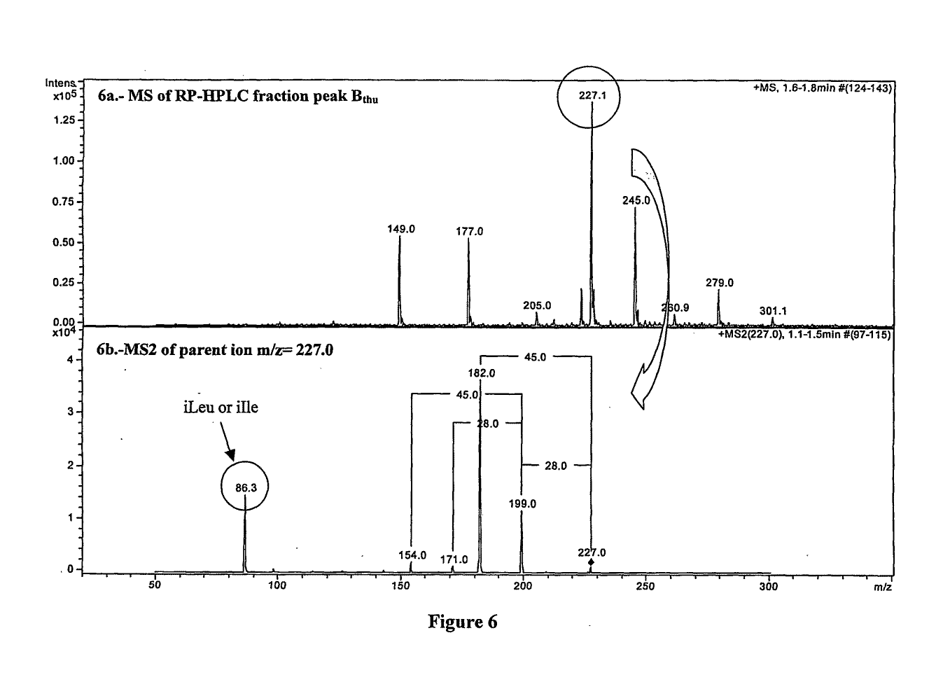

FIG. 6 illustrates the MS spectrum (a) and MSn spectra (b) of collected fraction of peak Bₜₕᵤ. Collected fraction was directly infused into the mass spectrometer and full scan MS acquired on line (FIG. 6a). Main m/z peak at 227.0±0.1 was isolated as parent ion and subjected to MSMS fragmentation giving rise to a daughter ion spectrum (FIG. 6b). Encircled peak m/z at 86.3 matches to immonium ion of leucine or isoleucine, respectively referred to as iLeu and iIle.

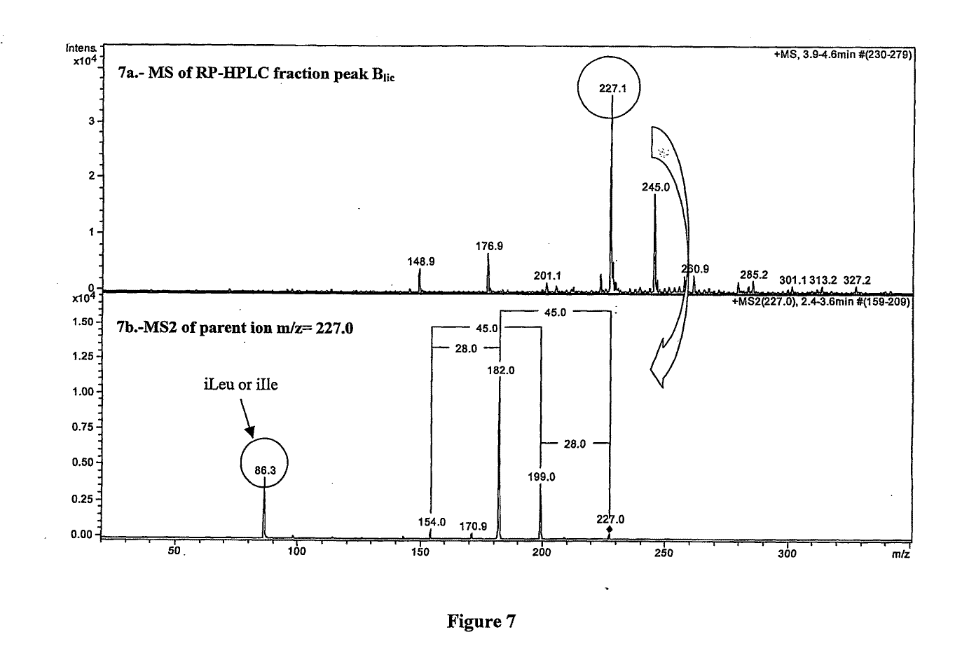

FIG. 7 illustrates the MS spectrum (a) and MSn spectra (b) of collected fraction of peak B_{lic}. Collected fraction was directly infused into the mass spectrometer and full scan MS acquired on line (FIG. 7a). Main m/z peak at 227.00±0.1 was isolated as parent ion and subjected to MSMS fragmentation giving rise to a daughter ion spectrum (FIG. 7b). Encircled peak m/z at 86.3 matches to immonium ion of leucine or isoleucine, respectively referred to as iLeu and iIle.

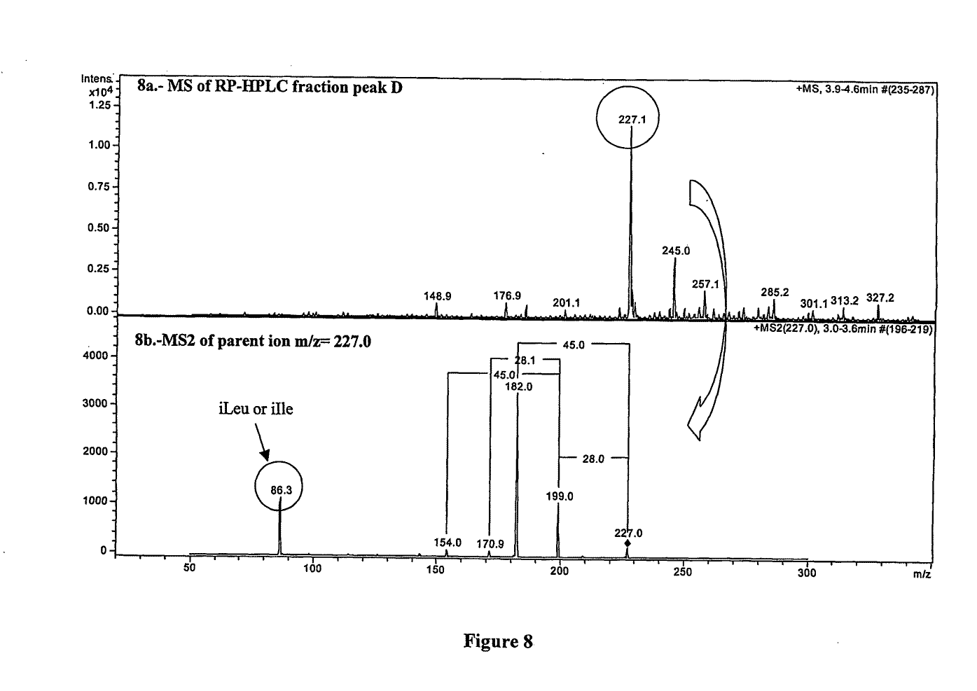

FIG. 8 illustrates the MS spectrum (a) and MSn spectra (b) of collected fraction of peak D. Collected fraction was directly infused into the mass spectrometer and full scan MS acquired on line (FIG. 8a). Main m/z peak at 227.00±0.1 was isolated as parent ion and subjected to MSMS fragmentation giving rise to a daughter ion spectrum (FIG. 8b). Encircled peak m/z at 86.3 matches to immonium ion of leucine or isoleucine, respectively referred to as iLeu and iIle.

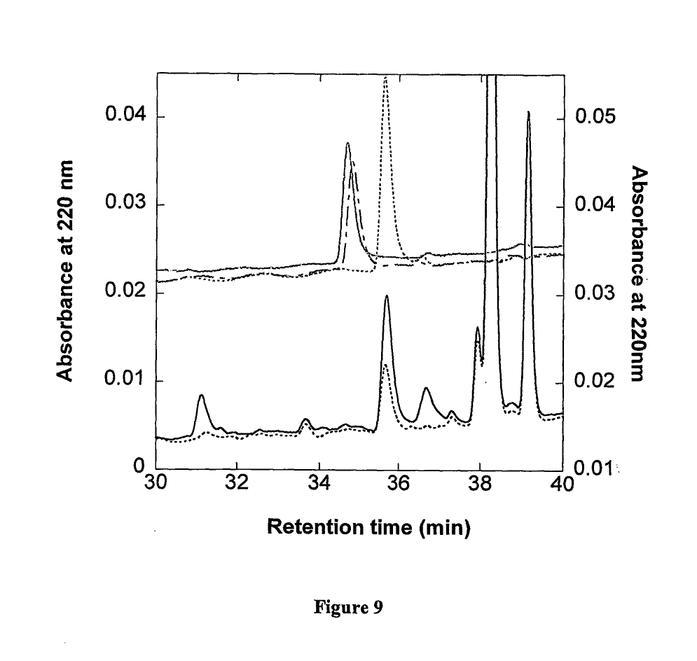

FIG. 9 illustrates the identification of the cyclodipeptides produced by YvmCsub-like and Plu0297 enzymes. E. coli BL21 AI cells expressing: [symbol] YvmC_{sub} from B. subtilis, and [symbol] Plu0297 from P. luminescens,

were grown in M9 medium.
The two culture supernatants were then analyzed by RP-HPLC. The cyclodipeptide standards: [symbol] cyclo(Ile-Ile), [symbol] cyclo(Ile-Leu), and [symbol] cyclo(Leu-Leu),
were also analyzed. The chromatograms were recorded at 220 nm (left scale for standards and right scale for assays).

It should be clearly understood, however, that these examples are given only by way of illustration of the subject of the invention, of which they in no way constitute a limitation.

### EXAMPLE 1

### Construction of an Escherichia coli Expression Vector Encoding the YvmC_{sub} Protein

As several homologues of YvmC from Bacilli are studied, these homologues were called YvmC_{sub} for the one from Bacillus subtilis sub. subtilis strain 168, YvmC_{lic} for the one from Bacillus licheniformis ATCC 14580 and YvmCₜₕᵤ for the one from Bacillus thuringiensis serovar israelensis 1884.

The expression vector encoding the said protein mentioned above were constructed using the Gateway™ cloning technology (Invitrogen). They were designed to express each protein as a cytoplasmic fusion protein carrying at its N-terminus end a (His)₆ tag, the translated sequence of the attB recombination site (necessary for cloning) and the TEV protease cleavage site.

The whole cloning strategy used to construct the expression vector encoding the YvmC_{sub} protein (Acc. n^{o} CAB15512) is shown in FIG. 2. First, the attB-flanked DNA suitable for recombinational cloning and encoding the YvmC_{sub} protein was obtained after three successive PCR. The YvmC_{sub} gene was amplified in the first PCR (PCR 1 in FIG. 2) using primers A and B as described in Table I and, as template, genomic DNA of Bacillus subtilis subsp. subtilis strain 168 whose genome has been completely sequenced (Acc. n^{o} AL009126). The PCR conditions were one initial denaturation step at 97° C. for 4 minutes followed by 25 cycles at 94° C. for one minute, 54° C. for one minute, 72° C. for 2 minutes, and one final extension step at 72° C. for 10 minutes. The reaction mixture (50 μl) comprised 1 μl of chromosomal DNA (25 ng/μl), 0.3 μl of each primer solution at 100 μM, 5 μl of 10×Pfu DNA polymerase buffer with MgSO₄ (provided by the Pfu DNA polymerase supplier), 0.1 μl of a mix of dNTPs 10 mM each and 1 μl of Pfu DNA polymerase (2.5 U/μl; Fermentas). The PCR product (herein after referred to as “PCR product 1”) was then purified using the “GFX PCR DNA and Gel Band Purification” kit (Amersham Biosciences) after electrophoresis in 1% agarose gel (16). The second PCR (PCR 2 in FIG. 2) enabled the addition of the sequence encoding the TEV protease cleavage site to the PCR product 1 5′ terminus and that of the attB2 encoding sequence to the 3′ terminus. PCR conditions were one initial denaturation step at 95° C. for 5 minutes followed by 30 cycles at 95° C. for 45 seconds, 50° C. for 45 seconds, 72° C. for 1.5 minutes, and one final extension step at 72° C. for 10 minutes. The reaction mixture (50 μl) comprised 5 ng PCR product 1, 0.4 μM of primers C and D as described in Table 1, 2.5 units Expand High Fidelity Enzyme mix (Roche), 1× Expand High Fidelity buffer with 1.5 mM MgCl₂ (Roche) and 200 μM each dNTP. After electrophoresis in 1% agarose gel and purification with the QIAquick Gel Extraction kit (Qiagen), the PCR product (hereinafter referred to as “PCR product 2”) was used for the third PCR (PCR 3 in FIG. 2) that enabled the addition to the PCR product 2 5′ terminus of the attB1 encoding sequence. PCR was carried out as described above using 5 ng PCR product 2 as a template and 0.4 μM of primers E and D as described in Table I. The resulting PCR product (hereinafter referred to as “PCR product 3”) was purified as previously described.

Second, the attB-flanked PCR product 3 was recombined with the pDONR™221 donor vector (Invitrogen) in BP Clonase™ reaction to generate the entry vector pENT-YvmC_{sub}. pENT-YvmC_{sub} was sequenced between the two-recombination sites using ABI PRISM 310 Genetic Analyzer (Applied Biosystem) and primers M13 forward, M13 reverse and F as described in Table 1. pENT-YvmC_{sub} and the commercial destination vector pDEST-17 (Invitrogen) were used in LR Clonase™ subcloning reaction to generate the expression vector pEXP-YvmC_{sub} (SEQ ID No 26) following the supplier recommendations. The recombination mixture was used for transformation of E. coli DH5α chemically competent cells and a positive clone was selected after analysis by colony PCR. The 50 μl reaction mix comprised a small amount of colony as a template, 200 μM each dNTP, 0.2 μM primer M13 forward and M13 reverse, 1× ThermoPol Reaction Buffer (New England Biolabs) and 1.25 unit Taq DNA Polymerase (New England Biolabs). The PCR conditions used were the following: one initial denaturation step at 95° C. for 5 minutes followed by 30 cycles at 92° C. for 30 seconds, 50° C. for 30 seconds, 72° C. for 2 minutes. Plasmid DNA was isolated from positive clones using the Wizard DNA Purification System (Promega) and conserved at −20° C.

TABLE I

Primers used to construct the expression vector pEXP-YvmC_{sub}.

| | |
|---|---|
| Name | Corresponding sequence (5′ to 3′) |

| | | |
|---|---|---|
| A | AAGTTTTAGGGGTGAATGAGATGACCGGAATGG | (SEQ ID No 9) |
| B | CGAAGCTTACTCCCCCTATCATCCTTCAGATGTGA | (SEQ ID No 10) |
| C | GGCTTCGAGAATCTTTATTTTCAGGGCACCGGAATGGTAACG | (SEQ ID No 11) |
| D | GGGGACCACTTTGTACAAGAAAGCTGGGTCCTTATCCTTCAGATGTGATCCG | (SEQ ID No: 12) |
| E | GGGGACAAGTTTGTACAAAAAAGCAGGCTTCGAGAATCTTTATTTTC | (SEQ ID No 13) |
| F | ATGAGGCGGCTAATCTTCTA | (SEQ ID No 14) |
| M13 Forward | GTAAACGACGGCCAG | (SEQ ID No 15) |
| M13 Reverse | CAGGAAACAGCTATGAC | (SEQ ID No 16) |

### EXAMPLE 2

### Construction of an Escherichia coli Expression Vector Encoding the Plu0297 Protein

The same cloning strategy was applied to construct the expression vector encoding Plu0297 (Acc. n^{o} Q7No 9M5). As template genomic DNA isolated from Photorhabdus luminescens subsp. laumondii TTO1 was used, whose genome has been sequenced (Acc. n^{o} BX470251) and the primers A, B, C, D, and E quoted in Table II to generate PCR product suitable for recombinational cloning. Then, pENT-Plu0297 and pEXP-Plu0297 were obtained after recombination with BP Clonase™ and LR Clonase™ as described above. DNA sequence of pENT-Plu0297 was verified by using the primers M13 forward and M13 reverse quoted in Table I and primer F described in Table II. Plasmid pEXP-Plu0297 (sequence SEQ ID No 27) was isolated from a positive clone obtained after colony PCR analysis as previously described.

TABLE II

Primers used to construct the expression vector pEXP-Plu0297.

| | |
|---|---|
| Name | Corresponding sequence (5′ to 3′) |

| | | |
|---|---|---|
| A | AACGAACAATCAAATATTATCAGCCCATTCAACATTGCTG | (SEQ ID No 17) |
| B | CGTATTAACTTTAAACGCAGTGACTATTTCATCAGACTGT | (SEQ ID No 18) |
| C | GGCTTCGAGAATCTTTATTTTCAGGGCCTGCACGAGAATTCACC | (SEQ ID No 19) |
| D | GGGGACCACTTTGTACAAGAAAGCTGGGTCCTTATAATTGAGTGACGATTCCG | (SEQ ID No 20) |
| E | GGGGACAAGTTTGTACAAAAAAGCAGGCTTCGAGAATCTTTATTTTC | (SEQ ID No 13) |
| F | GCCGGTGATAAAGTAACGAT | (SEQ ID No 21) |

### EXAMPLE 3

### Construction of an Escherichia coli Expression Vector Encoding the YvmCₜₕᵤ and YvmC_{lic} Proteins

For creating the expression vector encoding YvmCₜₕᵤ from Bacillus thuringiensis serovar israelensis 1884 and YvmC_{lic} (Acc. n^{o} AAU25020) from Bacillus licheniformis ATCC 14580, a slightly different strategy from the one used for YvmC_{sub} was used: the first PCR step (PCR1 in FIG. 2) was omitted, and DNA encoding YvmCₜₕᵤ, or YvmC_{lic} was directly amplified from corresponding chromosomal DNA by using oligonucleotides C and D described in Tables III and IV, respectively. Chromosomal DNA isolated from Bacillus thuringiensis israelensis 1884 for YvmCₜₕᵤ and chromosomal DNA isolated from Bacillus licheniformis ATCC 14580 were used, whose genome has been completely sequenced (Acc. n^{o} CP000002), for YvmC_{lic}. Constructions and verifications of the corresponding pENT and pEXP vectors (sequence SEQ ID No 28 for pEXP-YvmCₜₕᵤ and sequence SEQ ID No 29 for pEXP-YvmC_{lic}) were made as described for YvmC_{sub} and Plu0297.

TABLE III

Primers used to construct the expression vector pEXP-YvmCₜₕᵤ.

| | |
|---|---|
| Name | Corresponding sequence (5′ to 3′) |

| | | |
|---|---|---|
| C | GGCTTCGAGAATCTTTATTTTCAGGGCACGAATGCTATAGCGGTAAG | (SEQ ID No 22) |
| D | GGGGGGGGGACCACTTTGTACAAGAAAGCTGGGTCCTTAAGATAAATTTTCCATTTCTTGTACCA | (SEQ ID No 23) |
| E | GGGGACAAGTTTGTACAAAAAAGCAGGCTTCGAGAATCTTTATTTTC | (SEQ ID No 13) |

TABLE IV

Primers used to construct the expression vector pEXP-YvmC_{Ile}.

| | |
|---|---|
| Name | Corresponding sequence (5′ to 3′) |

| | | |
|---|---|---|
| C | GGCTTCGAGAATCTTTATTTTCAGGGCACAGAGCTTATAATGG | (SEQ ID No 24) |
| D | GGGGGGGGGACCACTTTGTACAAGAAAGCTGGGTCCTTATACTCGTTCCTCCTGCATGC | (SEQ ID No 25) |
| E | GGGGACAAGTTTGTACAAAAAAGCAGGCTTCGAGAATCTTTATTTTC | (SEQ ID No 13) |

### EXAMPLE 4

### Bacterial Cultures for Production of Cyclodipeptides

The same procedure was applied for expressing all the four proteins, YvmC_{sub}, YvmCₜₕᵤ, YvmC_{lic} and Plu0297. Bacteria were grown in minimal M9 medium (6 g/l Na₂HPO₄, 3 g/l KH₂PO₄, 0.5 g/l NaCl, 1 g/l NH₄Cl, 1 mM MgSO₄, 0.1 mM CaCl₂, 1 μg/ml thiamine and 0.5% glucose or glycerol) (Sambrook et al., (2001) Molecular Cloning: A Laboratory Manual, New York) supplemented with 1 ml of a vitamins solution and 2 ml of an oligoelements solution per liter of minimal medium. Vitamins solution contains 1.1 mg/l biotin, 1.1 mg/l folic acid, 110 mg/l para-amino-benzoic acid, 110 mg/l riboflavin, 220 mg/l pantothenic acid, 220 mg/l pyridoxine-HCl, 220 mg/l thiamine and 220 mg/l niacinamide in 50% ethanol. Oligoelements solution was made by diluting a FeCl₂-containing solution 50 fold in H₂O. The FeCl₂-containing solution contains for 100 ml: 8 ml concentrated HCl, 5 g FeCl₂.4H₂O, 184 mg CaCl₂.2H₂O, 64 mg H₃BO₃, 40 mg MnCl₂.4H₂O, 18 mg CoCl₂.6H₂O, 4 mg CuCl₂.2H₂O, 340 mg ZnCl₂, 605 mg Na₂MoO₄.2H₂O.

The whole process was performed using standard large-scale procedures and materials (e.g. Erlen Meyer flasks). 50 μl chemically competent BL21AI cells (Invitrogen) were transformed with 20 ng plasmid using standard heat-shock procedure (Sambrook et al., aforementioned). After 1 h outgrowth in SOC medium (Sambrook et al., aforementioned) at 37° C., bacteria were spread on LB plates containing 200 μg/ml ampicillin. A few colonies were picked up to inoculate M9 liquid medium supplemented with vitamins and oligoelements solutions containing 0.5% glucose and 200 μg/ml ampicillin. After overnight incubation at 37° C. with shaking, 500 μl of the starter cultures were used to inoculate 25 ml M9 minimal medium supplemented with vitamins and oligoelements solutions containing 0.5% glycerol and 200 μg/ml ampicillin. Cultures were grown at 37° C. until OD₆₀₀˜0.5 and 0.02% L-arabinose was added. Cultures were continued at 20° C. for 24 h. Cultures supernatants were collected after centrifugation at 2,500 g for 20 min and kept at −20° C.

As a control experiment, we used BL21AI cells transformed by pQE60 (Qiagen), an ampicillin resistance gene-carrying vector that expresses no cyclodipeptide synthesizing enzyme. Growth and supernatant collection were conducted as described above.

### EXAMPLE 5

### Comparison of Peptide Sequences

The sequences of the proteins YvmC_{sub}, YvmC_{lic}, YvmCₜₕᵤ and Plu0297 were compared. Two programs were used to measure the similarity/identity of those proteins one with another. The results are presented in Table V.

TABLE V
Comparison of sequences

| | YvmC_{sub} (248 aa) | YvmC_{lic} (249 aa) | YvmCₜₕᵤ (239 aa) | Plu0297 (234 aa) |
|---|---|---|---|---|
| YvmC_{sub} | 100/100 | Identities = | Identities = | Identities = 86/229 |
| 248 aa | | 174/247 (70%), | 147/238 (61%), | (37%), Positives = |
| | | Positives = | Positives = | 129/229 (56%), |
| | | 201/247 (81%), | 183/238 (76%), | Gaps = 3/229 (1%) |
| | | Gaps = 0/247 (0%) | Gaps = 0/238 (0%) | |
| | | 70.445% of identity | 61.765% of identity | 37.555% of identity |
| | | (86.640% similar) | (84.874% similar) | (68.996% similar) |
| | | in 247 aa overlap | in 238 aa overlap | in 229 aa overlap |
| YvmC_{lic} | | 100/100 | Identities = | Identities = 79/225 |
| 249 aa | | | 155/236 (65%), | (35%), Positives = |
| | | | Positives = | 133/225 (59%), |
| | | | 186/236 (78%), | Gaps = 3/225 (1%) |
| | | | Gaps = 0/236 (0%) | |
| | | | 65.678% of identity | 35.111% of identity |
| | | | (87.288% similar) | (70.667% similar) |
| | | | in 236 aa overlap | in 225 aa overlap |
| YvmCₜₕᵤ | | | 100/100 | Identities = 79/230 |
| 239 aa | | | | (34%), Positives = |
| | | | | 132/230 (57%), |
| | | | | Gaps = 3/230 (1%) |
| | | | | 34.348% of identity |
| | | | | (70.435% similar) |
| | | | | in 230 aa overlap |
| Plu0297 | | | | 100/100 |
| 234 aa | | | | |

Bold characters: result of comparisons with Blast (blast2seq, default parameters)/reference Tatusova and Madden, FEMS Microbiol. Lett., 1999, 174, 247-250).
Normal size characters: result of comparisons with SSEARCH (default parameters) (matrix BLOSUM50)/reference: Smith and Waterman, J. Mol. Biol., 1981, 147, 195-197; Pearson, Genomics, 1991, 11, 635-650).

### EXAMPLE 6

### Detection and Purification of a Cyclodipeptide in the Culture Media by HPLC Analysis

Culture supernatants (200 μl) were acidified down to pH=3 with concentrated trifluoroacetic acid and then analyzed by HPLC. Samples were loaded onto a C18 column (4.6×250 mm, Vydac) and eluted with a linear gradient from 0% to 55% acetonitrile/deionized water with 0.1% trifluoroacetic acid for 60 min (flow-rate, 1 ml/min). The elution was monitored between 220 and 500 nm using a diode array detector.

### EXAMPLE 7

### Identification of Cyclodipeptide Derivatives by MS and MSMS Analysis

HPLC-eluted fractions from culture supernatants (see above) were collected and analyzed by mass spectrometry using an ion trap mass analyzer Esquire HCT equipped with an orthogonal Atmospheric Pressure Interface-ElectroSpray Ionization (AP-ESI) source (Bruker Daltonik GmbH, Germany). The samples were directly infused into the mass spectrometer at a flow rate of 3 μl/min by means of a syringe pump. Nitrogen served as the drying and nebulizing gas while helium gas was introduced into the ion trap for efficient trapping and cooling of the ions generated by the ESI as well as for fragmentation processes. Ionization was carried out in positive mode with a nebulizing gas set at 9 psi, a drying gas set at 5 μl/min and a drying temperature set at 300° C. Ionization and mass analyses conditions (capillary high voltage, skimmer and capillary exit voltages and ions transfer parameters) were set for an optimal detection of compounds in the range of cyclodipeptides masses between 100 and 400 m/z. For MSMS experiments an isolation width of 1 mass unit was used for selecting the parent ion. Fragmentation amplitude was tuned until at least 90% of the isolated precursor ion was fragmented. Full scan MS and MSMS spectra were acquired using EsquireControl software and all data were processed using DataAnalysis software.

Different sources of cyclodipeptides were used as standards for mass and HPLC analyses. Cyclodipeptides cyclo(Leu-Leu) and cyclo(Phe-Leu) were respectively obtained from Sigma and Bachem. Cyclo(Ile-Leu) and cyclo(Ile-Ile) were chemically synthesised as described in Jeedigunta et al. (Tetrahedron, 2000, 56, 3303-3307).

### EXAMPLE 8

### Expression of YvmC_{sub}, YvmCₜₕᵤ, YvmC_{lic} or Plu0297 in E. coli Cytoplasm Leads to the Synthesis of Cyclodipeptides

The YvmC_{sub}-encoding gene of B. subtilis, the YvmCₜₕᵤ-encoding gene of B. thuringiensis israelensis 1884, the YvmC_{lic}-encoding gene of B. licheniformis and the Plu0297-encoding gene of P. luminescens were respectively cloned in expression vectors, their expression in E. coli cytoplasm was induced and the cyclodipeptide synthesis activity in vivo was searched. First, in each case, the synthesis of a protein whose N-terminal sequence corresponded to the one expected for the corresponding (His)₆-tagged protein was observed. Then, the hypothetical formation of cyclodipeptides was investigated by analyzing the culture supernatants of E. coli cells expressing YvmC_{sub}, YvmCₜₕᵤ, YvmC_{lic} or Plu0297.

HPLC analysis of the three culture supernatants of E. coli cells expressing YvmC_{sub}, YvmCₜₕᵤ, or YvmC_{lic} resulted in three similar 220 nm-chromatograms. The three chromatograms revealed the same three resolved peaks, respectively referred hereinafter to as “peak A”, peak B” and peak C”, that were not found in the culture supernatant of control cells which did not express any of those genes (FIG. 3). These three peaks hence corresponded to the elution of products whose synthesis was directly linked to the expression of YvmC_{sub}, YvmCₜₕᵤ or YvmC_{lic} in E. coli. Moreover, the similarity of the three 220 nm-chromatograms suggested that the three homologues from Bacillus synthesized the same compounds. Peaks A and C corresponded to the elution of compounds which do not relate to the instant invention. Peaks B were the major peaks and they were characterized by a retention time of 35.7 min. Peaks B of the three supernatants were collected for further analysis by mass spectrometry.

HPLC analysis of the culture supernatant of E. coli cells expressing Plu0297 showed only one resolved peak at 220 nm that was not found in the culture supernatant of control cells which did not express one of the genes of interest (FIG. 4). This peak, hereinafter referred to as “peak D”, corresponded to the elution of a product whose synthesis was directly linked to the expression of Plu0297 in E. coli. Peak D was characterized by a retention time of 35.7 min. Peak D was collected for further analysis by mass spectrometry.

### EXAMPLE 9

### Characterization of the Compounds Present in Peaks B and D by Mass Spectrometry

The first step in the identification of these compounds is the determination of their molecular masses. MS analyses were carried out in positive scanning mode for more sensitive detection. MS spectra of the eluted fractions corresponding to peaks B (hereinafter referred to as “peak B_{sub}” for analysis of the YvmC_{sub} supernatant, “peak Bₜₕᵤ” for analysis of the YvmCₜₕᵤ supernatant, and “peak B_{lic}” for analysis of the YvmC_{lic} supernatant) showed a major peak with the same m/z of 227.0±0.1 (FIGS. 5a, 6a, 7a). MS spectrum of the eluted fraction corresponding to peak D also showed a major peak with an m/z of 227.0±0.1 (FIG. 8a). We compared this m/z value to expected mass values of natural cyclodipeptides (quoted in Table VI). It matched to four possible cyclodipeptides: cyclo(Leu-Leu), cyclo(Ile-Ile), cyclo(Leu-Ile) and cyclo(Glu-Pro).

TABLE VI
Calculated monoisotopic mass (m/z) values of natural
cyclodipeptides under positive mode of ESI-MS.

| m/z of AA residue | Gly | Ala | Ser | Pro | Val | Thr | Cys | Ile | Leu | Asn | Asp |
|---|---|---|---|---|---|---|---|---|---|---|---|
| Gly | 115.0 | 129.1 | 145.1 | 155.1 | 157.1 | 159.1 | 161 | 171.1 | 171.1 | 172.1 | 173 |
| Ala | | 143.1 | 159.1 | 169.1 | 171.1 | 173.1 | 175 | 185.1 | 185.1 | 186.1 | 187.1 |
| Ser | | | 175.1 | 185.1 | 187.1 | 189.1 | 191 | 201.1 | 201.1 | 202.1 | 203.1 |
| Pro | | | | 195.1 | 197.1 | 199.1 | 201.1 | 211.1 | 211.1 | 212.1 | 213.1 |
| Val | | | | | 199.1 | 201.1 | 203.1 | 213.2 | 213.2 | 214.1 | 215.1 |
| Thr | | | | | | 203.1 | 205.1 | 215.1 | 215.1 | 216.1 | 217.1 |
| Cys | | | | | | | 207 | 217.1 | 217.1 | 218.1 | 219 |
| Ile | | | | | | | | 227.2 | 227.2 | 228.1 | 229.1 |
| Leu | | | | | | | | | 227.2 | 228.1 | 229.1 |
| Asn | | | | | | | | | | 229.1 | 230.1 |
| Asp | | | | | | | | | | | 231.1 |
| Gln | | | | | | | | | | | |
| Lys | | | | | | | | | | | |
| Glu | | | | | | | | | | | |
| Met | | | | | | | | | | | |
| His | | | | | | | | | | | |
| Phe | | | | | | | | | | | |
| Arg | | | | | | | | | | | |
| Tyr | | | | | | | | | | | |
| Trp | | | | | | | | | | | |

| m/z of AA residue | Gln | Lys | Glu | Met | His | Phe | Arg | Tyr | Trp |
|---|---|---|---|---|---|---|---|---|---|
| Gly | 186.1 | 186.1 | 187.1 | 189.1 | 195.1 | 205.1 | 214.1 | 221.1 | 244.1 |
| Ala | 200.1 | 200.1 | 201.1 | 203.1 | 209.1 | 219.1 | 228.1 | 235.1 | 258.1 |
| Ser | 216.1 | 216.1 | 217.1 | 219.1 | 225.1 | 235.1 | 244.1 | 251.1 | 274.1 |
| Pro | 226.1 | 226.1 | 227.1 | 229.1 | 235.1 | 245.1 | 254.2 | 261.1 | 284.1 |
| Val | 228.1 | 228.2 | 229.1 | 231.1 | 237.1 | 247.1 | 256.2 | 263.1 | 286.1 |
| Thr | 230.1 | 230.1 | 231.1 | 233.1 | 239.1 | 249.1 | 258.1 | 265.1 | 288.1 |
| Cys | 232.1 | 232.1 | 233.1 | 235 | 241.1 | 251.1 | 260.1 | 267.1 | 290.1 |
| Ile | 242.1 | 242.2 | 243.1 | 245.1 | 251.1 | 261.2 | 270.2 | 277.1 | 300.2 |
| Leu | 242.1 | 242.2 | 243.1 | 245.1 | 251.1 | 261.2 | 270.2 | 277.1 | 300.2 |
| Asn | 243.1 | 243.1 | 244.1 | 246.1 | 252.1 | 262.1 | 271.1 | 278.1 | 301.1 |
| Asp | 244.1 | 244.1 | 245.1 | 247.1 | 253.1 | 263.1 | 272.1 | 279.1 | 302.1 |
| Gln | 257.1 | 257.2 | 258.1 | 260.1 | 266.1 | 276.1 | 285.2 | 292.1 | 315.1 |
| Lys | | 257.2 | 258.1 | 260.1 | 266.2 | 276.2 | 285.2 | 292.2 | 315.2 |
| Glu | | | 259.1 | 261.1 | 267.1 | 277.1 | 286.1 | 293.1 | 316.1 |
| Met | | | | 263.1 | 269.1 | 279.1 | 288.1 | 295.1 | 318.1 |
| His | | | | | 275.1 | 285.1 | 294.2 | 301.1 | 324.1 |
| Phe | | | | | | 295.1 | 304.2 | 311.1 | 334.1 |
| Arg | | | | | | | 313.2 | 320.2 | 343.2 |
| Tyr | | | | | | | | 327.1 | 350.1 |
| Trp | | | | | | | | | 373.2 |

In a second step, MSMS experiments were performed in order to elucidate the chemical structure of these compounds. As already experimented on different commercial or home-made synthetic cyclodipeptides (data not shown) and also observed on cyclodipeptides daughter ions spectra published elsewhere (Chen et al., Eur Food Research Technology, 2004, 218, 589-597; Stark et al., J Agric Food Chem, 2005, 53, 7222-7231), cyclodipeptide derivatives are fragmented following a characteristic pattern: (i) a sequence of neutral losses which results from cleavages of the diketopiperazine ring on either sides of the carbonyl group (loss of 28 uma corresponding to the departure of C═O group) or of the amido group (loss of 45 corresponding to CONH₃) and (ii) the presence of m/z peaks of the so-called immonium ions and of their related ions (Roepstorff et al., Biomed Mass Spectrom, 1984, 11, 601; Johnson et al., Anal. Chem., 1987, 59, 2621-2625) which enable to identify amino acyl residues (Table VII).

TABLE VII
Immonium and related ion masses m/z used for the identification of the
cyclodipeptides

| Residue | Immonium ion* | Related ions* |
|---|---|---|
| Gly | 30 | |
| Ala | 44 | |
| Ser | 60 | |
| Pro | 70 | |
| Val | 72 | 41, 55, 69 |
| Thr | 74 | |
| Cys | 76 | |
| Ile | 86 | 44, 72 |
| Leu | 86 | 44, 72 |
| Asn | 87 | 70 |
| Asp | 88 | 70 |
| Gln | 101 | 56, 84, 129 |
| Lys | 101 | 70, 84, 112, 129 |
| Glu | 102 | |
| Met | 104 | 61 |
| His | 110 | 82, 121, 123, 138, 166 |
| Phe | 120 | 91 |
| Arg | 129 | 59, 70, 73, 87, 100, 112 |
| Tyr | 136 | 91, 107 |
| Trp | 159 | 77, 117, 130,132, 170, 171 |

* Bold face indicates strong signals,
italic indicates weak.
(Reference for this table: Falick et al., J. Am. Soc. Mass Spectrom., 1993, 4, 882-893; Papayannopoulos, Mass Spectrom. Rev., 1995, 14, 49-73)

“Peak B_{sub}”, “peak Bₜₕᵤ”, “peak B_{lic},”, and “peak D” fractions were then subjected to fragmentations in the ion trap mass spectrometer by isolating m/z peak at 227.0±0.1 as parent ion. As shown in every daughter ions spectrum, MSMS fragmentation of m/z peak at 227.0±0.1 from compounds B_{sub}, Bₜₕᵤ, B_{lic}, and D (FIGS. 5b, 6b, 7b and 8b respectively) produces both the characteristic signature of fragmentation of diketopiperazine ring with the neutral losses of 28 and 45 and a m/z peak at 86 that could be attributed to the leucine residue or its isomass compound isoleucine. These results enabled to attribute the major m/z peak in HPLC-fractions B_{sub}, Bₜₕᵤ, B_{lic} and D to either cyclo(Leu-Leu) or cyclo(Leu-Ile) or cyclo(Ile-Ile).

### EXAMPLE 10

### Recombinant E. coli Expressing YvmC_{sub}, YvmCₜₕᵤ, YvmC_{lic}, and Plu0297 Produce Cyclo(Leu-Leu)

As observed previously, mass spectrometry analysis cannot differentiate cyclodipeptides containing isoleucine from those containing leucine residues. To check the chromatographic behaviours of the three different cyclodipeptides cyclo(Leu-Leu), cyclo(Leu-Ile) and cyclo(Ile-Ile) were checked in order to detect differences that could lead us to the final identification of the product synthesized by YvmC_{sub}, YvmCₜₕᵤ, YvmC_{lic} and Plu0297. HPLC-analysis at 220 nm of the three reference compounds showed that they display different retention times as cyclo(Ile-Ile) is first eluted at 34.7 min, cyclo(Ile-Leu) at 34.9 min and finally cyclo(Leu-Leu) at 35.7 min (FIG. 9). The cyclodipeptides produced by YvmC_{sub} and Plu0297 were then analysed by HPLC. The 220 nm-chromatograms showed that the cyclodipeptides produced by YvmC_{sub} and Plu0297 and characterized by m/z values of 227.0±0.1 (FIGS. 5a and 8a) were eluted with a retention time around 35.7 min that is similar to that of cyclo(Leu-Leu) (FIG. 9). This result was confirmed by performing co-injections of reference cyclo(Leu-Leu) and culture supernatants of cells expressing YvmC_{sub} and Plu0297. Consequently, the compounds eluted in peak B_{sub} (FIGS. 3 & 4) and peak D (FIG. 5) correspond to cyclo(Leu-Leu). Moreover, peaks Bₜₕᵤ, B_{lic}, being characterized by the same retention time than that of peak B_{sub}, the corresponding eluted compounds are also cyclo(Leu-Leu).

Thus expression of YvmC_{sub}, YvmCₜₕᵤ, YvmC_{lic} or Plu0297 enzymes in E. coli leads to the synthesis of cyclo(Leu-Leu) cyclodipeptides found in the culture medium, demonstrating that Plu0297 and the three YvmC-like proteins are cyclo(Leu-Leu)-synthetases that can be produced in active forms in E. coli.

### EXAMPLE 11

### In Vitro Production of Cyclo(Leu-Leu) by the Purified YvmC_{sub} Protein

Production of the Purified YvmC_{sub} Protein

Bacterial culture for production of the YvmC_{sub} protein was performed as already described in Example 4, except that minimal medium was replaced by LB medium (Sambrook et al., aforementioned). After induction with 0.02% arabinose, the culture was continued at 20° C. for 12 h. The bacterial cells were harvested by centrifugation at 4,000 g for 20 min and frozen at −80° C. Then, bacterial cells were thawed and resuspended in 1.5 ml of an extraction buffer composed of 100 mM Tris-HCl pH 8, 150 mM NaCl and 5% glycerol. Cells were broken using an Eaton press and centrifuged at 20,000 g and 4° C. for 20 min. The resulting supernatant containing the soluble proteins was loaded onto a Ni²⁺-column (HisTrap HP from Amersham) equilibrated with a buffer composed of 100 mM Tris-HCl pH 8, 150 mM NaCl. The column was washed with the same buffer and submitted to a linear gradient of imidazole (from 0 to 1 μM imidazole at pH 8). The YvmC_{sub} protein was eluted at around 250 mM imidazole. The purified YvmC_{sub} protein was then washed (to eliminate imidazole) and concentrated using a Vivaspin concentrator (Vivascience).

Preparation of the Soluble Cell Extract Used for Supplementation

Bacteria transformed with the empty vector pQE60 (Qiagen) were cultivated and broken as previously described. The broken cells were centrifuged at 20,000 g and 4° C. for 20 min. The resulting supernatant corresponds to a soluble extract of E. coli cells, which does not contain cyclodipeptide synthetase.

In Vitro Production of Cyclo(Leu-Leu)

A 215 μl-reaction mixture comprising 6.0 mM Phe, 7.6 mM Leu, 10 mM ATP, 20 mM MgCl₂ and 25 μM of the purified YvmC_{sub} protein was supplemented with 115 μl of the previously described soluble cell extract. This mixture was incubated at 30° C. for 12 h. The reaction was stopped by adding TFA and submitted to a centrifugation at 20,000 g for 20 min. The supernatant was then analyzed by HPLC and HPLC-eluted fractions were characterized by mass spectrometry as described in Examples 6 and 7. As a control, the same experiment was performed under similar conditions except that the purified YvmC_{sub} was omitted.

The results clearly showed that the incubated mixture comprising the YvmC_{sub} protein contains cyclo(Leu-Leu) (an HPLC-eluted fraction at a retention time of 35.7 min with mass characteristics similar to that shown in FIG. 5) whereas the incubated mixture devoid of the YvmC_{sub} protein contains no cyclodipeptide. This demonstrates that the formation of cyclo(Leu-Leu) can be performed in vitro with a purified cyclo(Leu-Leu) synthetase.

The procedure described for the YvmC_{sub} protein can be applied to YvmCₜₕᵤ, YvmC_{lic} and Plu0297.

1.-3. (canceled)

4. An isolated cyclodipeptide synthetase, which:
a) has an ability to produce cyclo(Leu-Leu) cyclodipeptide from two Leu amino acids, and
b) comprises a polypeptide sequence having at least 34% of identity or at least 56% of similarity, with at least one of the polypeptides selected from the group consisting of sequences SEQ ID No 2, SEQ ID No 4, SEQ ID No 6 and SEQ ID No 8.

5.-6. (canceled)

7. An isolated polynucleotide selected from the group consisting of:
a) a polynucleotide encoding a cyclodipeptide synthetase as defined in claim 4;
b) a complementary polynucleotide of the polynucleotide a);
c) a polynucleotide which hybridises to polynucleotide a) or b) under stringent hybridization conditions.

8. The isolated polynucleotide according to claim 7, which is selected from the group consisting of the polynucleotides of sequences SEQ ID No 1, SEQ ID No 3, SEQ ID No 5 and SEQ ID No 7.

9. A recombinant vector comprising a polynucleotide as defined in claim 6.

10. A modified host cell comprising a polynucleotide as defined in claim 6.

11.-12. (canceled)

13. A method for the synthesis of a cyclo(Leu-Leu) cyclodipeptide, which comprises the steps of:
(1) incubating leucine, under suitable conditions, with at least one cyclodipeptide synthetase as defined in claim 4, and
(2) recovering the cyclo(Leu-Leu) cyclodipeptide thus obtained.

14. A method for the synthesis of α,β-dehydrogenated cyclo (Leu-Leu) cyclodipeptide, which comprises the steps of:
(1) incubating leucine, under suitable conditions, with a cyclodipeptide synthetase enzyme as defined in claim 4, and a purified CDO, and
(2) recovering the α,β-dehydrogenated cyclodipeptide.

15. The method according to claim 13, wherein step (1) is performed in presence of Leu at a concentration between 0.1 mM and 100 mM, said cyclodipeptide synthetase at a concentration between 0.1 mM and 100 μM, in a buffer at pH of between 6 and 8, and containing a soluble extract of prokaryote cells such as E. coli or Streptomyces cells which does not produce cyclodipeptide synthetase.

16. The method according to claim 14, which further comprises, prior or simultaneously with step (1), a step synthesizing said cyclodipeptide synthetase using a polynucleotide as defined in claim 7.

17. A method for producing cyclo(Leu-Leu) cyclodipeptide which comprises the steps of:
(1) culturing a host cell according to claim 10, in suitable culture conditions for said host cell, and
(2) recovering the cyclo(Leu-Leu) cyclodipeptide from the culture medium.

18. A method for the synthesis of α,β-dehydrogenated cyclo(Leu-Leu) cyclodipeptide, which comprises the following steps:
(1) culturing a modified host cell according to claim 10 in appropriate culture conditions for said host cell,
(2) incubating the cyclo(Leu-Leu) cyclodipeptide obtained from step (1′) with a purified CDO, and
(3) recovering the α,β-dehydrogenated cyclo(Leu-Leu) cyclodipeptide derivative from the culture medium.

19. α,β-dehydrogenated cyclo(Leu-Leu) cyclodipeptides, which are selected from the group consisting of cyclo(ΔLeu-Leu)±cyclo(ΔLeu-ΔLeu), and a mixture thereof.

20. The method according to claim 15, wherein said Leu is in a concentration of 1 mM to 10 mM.

21. The method according to claim 15, wherein said cyclodipeptide synthetase is in a concentration of 1 μM to 100 μM.

22. The method according to claim 15, wherein said prokaryote cells are E. coli.

23. The method according to claim 15, wherein said prokaryote cells are Streptomyces cells.


SEQUENCE LISTING (30 sequences)

SEQ ID NO: 1 - DNA, 745 bp, Bacillus subtilis
  CDS (1)..(744)
atgaccggaatggtaacggaaagaaggtctgtgcattttattgctgaggcattaacagaaaactgcagagaaatatttgaacggcgcaggcatgttttggtggggatcagcccatttaacagcaggttttcagaggattatatttacagattaattggatgggcgaaagctcaatttaaaagcgtttcagttttacttgcagggcatgaggcggctaatcttctagaagcgcttggaactccgagaggaaaggctgaacgaaaagtaaggaaagaggtatcacgaaacaggagatttgcagaaagagcccttgtggctcatggcggggatccgaaggcgattcatacattttctgattttatagataacaaagcctaccagctgttgagacaagaagttgaacatgcattttttgagcagcctcattttcgacatgcttgtttggacatgtctcgtgaagcgataatcgggcgtgcgcggggcgtcagtttgatgatggaagaagtcagtgaggatatgctgaatttggctgtggaatatgtcatagctgagctgccgttttttatcggagctccggatattttagaggtggaagagacactccttgcttatcatcgtccgtggaagctgggtgagaagatcagtaaccatgaattttctatttgtatgcggccgaatcaagggtatctcattgtacaggaaatggcgcagatgctttctgagaaacggatcacatctgaaggat

SEQ ID NO: 2 - protein, 248 aa, Bacillus subtilis
MTGMVTERRSVHFIAEALTENCREIFERRRHVLVGISPFNSRFSEDYIYRLIGWAKAQFKSVSVLLAGHEAANLLEALGTPRGKAERKVRKEVSRNRRFAERALVAHGGDPKAIHTFSDFIDNKAYQLLRQEVEHAFFEQPHFRHACLDMSREAIIGRARGVSLMMEEVSEDMLNLAVEYVIAELPFFIGAPDILEVEETLLAYHRPWKLGEKISNHEFSICMRPNQGYLIVQEMAQMLSEKRITSEG

SEQ ID NO: 3 - DNA, 705 bp, Photorhabdus luminescens
  CDS (1)..(705)
atgctgcacgagaattcaccatcatttactgtccaaggtgaaacctctcgttgtgaccaaattattcaaaaaggtgatcacgcgctaatagggataagcccctttaactcgcgtttttcaaaagactatgtagtggaccttattcagtggtcaagtcattatttccgacaagtcgacatattattaccttgtgaacgtgaagcttcacgccttttagtcgctagtggaattgataatgttaaagctatcaaaaaaacacatcgcgaaattagacgtcatttacgtaaccttgattatgttatttccacagcaacattgaaaagtaagcaaatcagagtcatccaatttagtgacttttcactaaaccatgactaccaatctcttaaaacacaagttgaaaacgcgtttaatgaatcagaatcttttaaaaaaagctgtcttgatatgtcctttcaagccataaaagggcgactaaaaggtactgggcaatactttggtcaaattgacctacaattagtatataaagcgttgccatatattttcgctgaaattcctttttacctcaatacccctcgattacttggggtaaagtattctacgttactttatcaccgcccttggtcaatcggaaaagggttatttaacggtagttatcctatacaagtagcagataaacaaagttacggaatcgtcactcaattataa

SEQ ID NO: 4 - protein, 234 aa, Photorhabdus luminescens
MLHENSPSFTVQGETSRCDQIIQKGDHALIGISPFNSRFSKDYVVDLIQWSSHYFRQVDILLPCEREASRLLVASGIDNVKAIKKTHREIRRHLRNLDYVISTATLKSKQIRVIQFSDFSLNHDYQSLKTQVENAFNESESFKKSCLDMSFQAIKGRLKGTGQYFGQIDLQLVYKALPYIFAEIPFYLNTPRLLGVKYSTLLYHRPWSIGKGLFNGSYPIQVADKQSYGIVTQL

SEQ ID NO: 5 - DNA, 720 bp, Bacillus thuringiensis
  CDS (1)..(720)
atgacgaatgctatagcggtaagaaatgtacgaaagtttagttctcaacccttatctactaattgtgctgaaatattaaaacgtagtaagcatgcaataataggtattagtccgtttaatagtagattttctgatgaatatattaatagactcattgaatgggcattacatacttttgatgatgttagtgttttattagctggaaaagaagctgcaaatttacttgaggctctaggaacaccaaaaggtaaagcggaaagaaaagttaggaaagaagtatctcgaaatagaagatcagctgaaaaggcacttaaagagcatggtggtaatgtaaatgctatccatactttttctgattttaatgacaacaatgcatatagctgcatgagggcagaagcagaacatatttttttaagcgaaactgtttttcgaaatgcttgcttagaaatgtcacatgcagccattttaggtagggcaaggggtactaatatagatattgatcaaatatcaaatgacatgctaaatatcgcagtagaatatgtaattgcagaactcccatttttcattggtggagctgaaattttaggaactcaagaagctgtacttatttatcataaaccatgggagcttggtgaacagatagttagaaatgatttttctatcaggatgaaaccaaatcaaggatatttaatggtacaagacatggaaaatttatcttaa

SEQ ID NO: 6 - protein, 239 aa, Bacillus thuringiensis
MTNAIAVRNVRKFSSQPLSTNCAEILKRSKHAIIGISPFNSRFSDEYINRLIEWALHTFDDVSVLLAGKEAANLLEALGTPKGKAERKVRKEVSRNRRSAEKALKEHGGNVNAIHTFSDFNDNNAYSCMRAEAEHIFLSETVFRNACLEMSHAAILGRARGTNIDIDQISNDMLNIAVEYVIAELPFFIGGAEILGTQEAVLIYHKPWELGEQIVRNDFSIRMKPNQGYLMVQDMENLS

SEQ ID NO: 7 - DNA, 748 bp, Bacillus licheniformis
  CDS (1)..(747)
atgacagagcttataatggagagcaaacaccagctattcaaaaccgaaactcttacccaaaactgcaatgaaatattaaaacgcagacgccatgttctcgtcggcatcagcccgtttaacagccgattttccgaagattatattcatcggcttatcgcctgggccgtccgtgagtttcagagtgtatccgtgcttttggcgggaaaggaagctgccaaccttctcgaagcgctcggcaccccacatgggaaggccgaacggaaagtcaggaaagaagtctcgcggaaccggagattcgctgaaaaggcgttggaagcgcatggcggaaatcccgaggacatccatacattttccgatttcgcgaaccagaccgcataccggaatttgcggatggaagtcgaagctgcctttttcgaccagacgcattttcgcaatgcctgcctggagatgtcgcatgcggctatcctcggacgggcccggggcactcggatggatgtcgtggaagtcagcgcagacatgctggagctggctgttgaatacgtcatcgctgaacttccgtttttcatcgccgcccctgatattttaggcgtcgaagagacgcttcttgcttatcaccggccatggaagctcggcgaacagatctcccgtaatgaatttgccgtcaaaatgcggccgaatcaaggatatctcatggtttccgaagcggacgaaagggtggaatctaaaagcatgcaggaggaacgagtat

SEQ ID NO: 8 - protein, 249 aa, Bacillus licheniformis
MTELIMESKHQLFKTETLTQNCNEILKRRRHVLVGISPFNSRFSEDYIHRLIAWAVREFQSVSVLLAGKEAANLLEALGTPHGKAERKVRKEVSRNRRFAEKALEAHGGNPEDIHTFSDFANQTAYRNLRMEVEAAFFDQTHFRNACLEMSHAAILGRARGTRMDVVEVSADMLELAVEYVIAELPFFIAAPDILGVEETLLAYHRPWKLGEQISRNEFAVKMRPNQGYLMVSEADERVESKSMQEERV

SEQ ID NO: 9 - DNA, 33 bp, Artificial Sequence
  Description of Artificial Sequence Synthetic primer
aagttttaggggtgaatgagatgaccggaatgg

SEQ ID NO: 10 - DNA, 35 bp, Artificial Sequence
  Description of Artificial Sequence Synthetic primer
cgaagcttactccccctatcatccttcagatgtga

SEQ ID NO: 11 - DNA, 42 bp, Artificial Sequence
  Description of Artificial Sequence Synthetic primer
ggcttcgagaatctttattttcagggcaccggaatggtaacg

SEQ ID NO: 12 - DNA, 52 bp, Artificial Sequence
  Description of Artificial Sequence Synthetic primer
ggggaccactttgtacaagaaagctgggtccttatccttcagatgtgatccg

SEQ ID NO: 13 - DNA, 47 bp, Artificial Sequence
  Description of Artificial Sequence Synthetic primer
ggggacaagtttgtacaaaaaagcaggcttcgagaatctttattttc

SEQ ID NO: 14 - DNA, 20 bp, Artificial Sequence
  Description of Artificial Sequence Synthetic primer
atgaggcggctaatcttcta

SEQ ID NO: 15 - DNA, 15 bp, Artificial Sequence
  Description of Artificial Sequence Synthetic primer
gtaaacgacggccag

SEQ ID NO: 16 - DNA, 17 bp, Artificial Sequence
  Description of Artificial Sequence Synthetic primer
caggaaacagctatgac

SEQ ID NO: 17 - DNA, 40 bp, Artificial Sequence
  Description of Artificial Sequence Synthetic primer
aacgaacaatcaaatattatcagcccattcaacattgctg

SEQ ID NO: 18 - DNA, 40 bp, Artificial Sequence
  Description of Artificial Sequence Synthetic primer
cgtattaactttaaacgcagtgactatttcatcagactgt

SEQ ID NO: 19 - DNA, 44 bp, Artificial Sequence
  Description of Artificial Sequence Synthetic primer
ggcttcgagaatctttattttcagggcctgcacgagaattcacc

SEQ ID NO: 20 - DNA, 53 bp, Artificial Sequence
  Description of Artificial Sequence Synthetic primer
ggggaccactttgtacaagaaagctgggtccttataattgagtgacgattccg

SEQ ID NO: 21 - DNA, 20 bp, Artificial Sequence
  Description of Artificial Sequence Synthetic primer
gccggtgataaagtaacgat

SEQ ID NO: 22 - DNA, 47 bp, Artificial Sequence
  Description of Artificial Sequence Synthetic primer
ggcttcgagaatctttattttcagggcacgaatgctatagcggtaag

SEQ ID NO: 23 - DNA, 65 bp, Artificial Sequence
  Description of Artificial Sequence Synthetic primer
gggggggggaccactttgtacaagaaagctgggtccttaagataaattttccatttcttgtacca

SEQ ID NO: 24 - DNA, 43 bp, Artificial Sequence
  Description of Artificial Sequence Synthetic primer
ggcttcgagaatctttattttcagggcacagagcttataatgg

SEQ ID NO: 25 - DNA, 59 bp, Artificial Sequence
  Description of Artificial Sequence Synthetic primer
gggggggggaccactttgtacaagaaagctgggtccttatactcgttcctcctgcatgc

SEQ ID NO: 26 - DNA, 5468 bp, Artificial Sequence
  Description of Artificial Sequence Synthetic polynucleotide
agatctcgatcccgcgaaattaatacgactcactatagggagaccacaacggtttccctctagaaataattttgtttaactttaagaaggagatatacatatgtcgtactaccatcaccatcaccatcacctcgaatcaacaagtttgtacaaaaaagcaggcttcgagaatctttattttcagggcaccggaatggtaacggaaagaaggtctgtgcattttattgctgaggcattaacagaaaactgcagagaaatatttgaacggcgcaggcatgttttggtggggatcagcccatttaacagcaggttttcagaggattatatttacagattaattggatgggcgaaagctcaatttaaaagcgtttcagttttacttgcagggcatgaggcggctaatcttctagaagcgcttggaactccgagaggaaaggctgaacgaaaagtaaggaaagaggtatcacgaaacaggagatttgcagaaagagcccttgtggctcatggcggggatccgaaggcgattcatacattttctgattttatagataacaaagcctaccagctgttgagacaagaagttgaacatgcattttttgagcagcctcattttcgacatgcttgtttggacatgtctcgtgaagcgataatcgggcgtgcgcggggcgtcagtttgatgatggaagaagtcagtgaggatatgctgaatttggctgtggaatatgtcatagctgagctgccgttttttatcggagctccggatattttagaggtggaagagacactccttgcttatcatcgtccgtggaagctgggtgagaagatcagtaaccatgaattttctatttgtatgcggccgaatcaagggtatctcattgtacaggaaatggcgcagatgctttctgagaaacggatcacatctgaaggataaggacccagctttcttgtacaaagtggttgattcgaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 27 - DNA, 5426 bp, Artificial Sequence
  Description of Artificial Sequence Synthetic polynucleotide
agatctcgatcccgcgaaattaatacgactcactatagggagaccacaacggtttccctctagaaataattttgtttaactttaagaaggagatatacatatgtcgtactaccatcaccatcaccatcacctcgaatcaacaagtttgtacaaaaaagcaggcttcgagaatctttattttcagggcctgcacgagaattcaccatcatttactgtccaaggtgaaacctctcgttgtgaccaaattattcaaaaaggtgatcacgcgctaatagggataagcccctttaactcgcgtttttcaaaagactatgtagtggaccttattcagtggtcaagtcattatttccgacaagtcgacatattattaccttgtgaacgtgaagcttcacgccttttagtcgctagtggaattgataatgttaaagctatcaaaaaaacacatcgcgaaattagacgtcatttacgtaaccttgattatgttatttccacagcaacattgaaaagtaagcaaatcagagtcatccaatttagtgacttttcactaaaccatgactaccaatctcttaaaacacaagttgaaaacgcgtttaatgaatcagaatcttttaaaaaaagctgtcttgatatgtcctttcaagccataaaagggcgactaaaaggtactgggcaatactttggtcaaattgacctacaattagtatataaagcgttgccatatattttcgctgaaattcctttttacctcaatacccctcgattacttggggtaaagtattctacgttactttatcaccgcccttggtcaatcggaaaagggttatttaacggtagttatcctatacaagtagcagataaacaaagttacggaatcgtcactcaattataaggacccagctttcttgtacaaagtggttgattcgaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 28 - DNA, 5441 bp, Artificial Sequence
  Description of Artificial Sequence Synthetic polynucleotide
agatctcgatcccgcgaaattaatacgactcactatagggagaccacaacggtttccctctagaaataattttgtttaactttaagaaggagatatacatatgtcgtactaccatcaccatcaccatcacctcgaatcaacaagtttgtacaaaaaagcaggcttcgagaatctttattttcagggcacgaatgctatagcggtaagaaatgtacgaaagtttagttctcaacccttatctactaattgtgctgaaatattaaaacgtagtaagcatgcaataataggtattagtccgtttaatagtagattttctgatgaatatattaatagactcattgaatgggcattacatacttttgatgatgttagtgttttattagctggaaaagaagctgcaaatttacttgaggctctaggaacaccaaaaggtaaagcggaaagaaaagttaggaaagaagtatctcgaaatagaagatcagctgaaaaggcacttaaagagcatggtggtaatgtaaatgctatccatactttttctgattttaatgacaacaatgcatatagctgcatgagggcagaagcagaacatatttttttaagcgaaactgtttttcgaaatgcttgcttagaaatgtcacatgcagccattttaggtagggcaaggggtactaatatagatattgatcaaatatcaaatgacatgctaaatatcgcagtagaatatgtaattgcagaactcccatttttcattggtggagctgaaattttaggaactcaagaagctgtacttatttatcataaaccatgggagcttggtgaacagatagttagaaatgatttttctatcaggatgaaaccaaatcaaggatatttaatggtacaagacatggaaaatttatcttaaggacccagctttcttgtacaaagtggttgattcgaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 29 - DNA, 5471 bp, Artificial Sequence
  Description of Artificial Sequence Synthetic polynucleotide
agatctcgatcccgcgaaattaatacgactcactatagggagaccacaacggtttccctctagaaataattttgtttaactttaagaaggagatatacatatgtcgtactaccatcaccatcaccatcacctcgaatcaacaagtttgtacaaaaaagcaggcttcgagaatctttattttcagggcacagagcttataatggagagcaaacaccagctattcaaaaccgaaactcttacccaaaactgcaatgaaatattaaaacgcagacgccatgttctcgtcggcatcagcccgtttaacagccgattttccgaagattatattcatcggcttatcgcctgggccgtccgtgagtttcagagtgtatccgtgcttttggcgggaaaggaagctgccaaccttctcgaagcgctcggcaccccacatgggaaggccgaacggaaagtcaggaaagaagtctcgcggaaccggagattcgctgaaaaggcgttggaagcgcatggcggaaatcccgaggacatccatacattttccgatttcgcgaaccagaccgcataccggaatttgcggatggaagtcgaagctgcctttttcgaccagacgcattttcgcaatgcctgcctggagatgtcgcatgcggctatcctcggacgggcccggggcactcggatggatgtcgtggaagtcagcgcagacatgctggagctggctgttgaatacgtcatcgctgaacttccgtttttcatcgccgcccctgatattttaggcgtcgaagagacgcttcttgcttatcaccggccatggaagctcggcgaacagatctcccgtaatgaatttgccgtcaaaatgcggccgaatcaaggatatctcatggtttccgaagcggacgaaagggtggaatctaaaagcatgcaggaggaacgagtataaggacccagctttcttgtacaaagtggttgattcgaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 30 - protein, 6 aa, Artificial Sequence
  Description of Artificial Sequence Synthetic 6xHis tag
HHHHHH